(19) United States
(12) Patent Application Publication
(10) Pub. No.: US 2025/0114116 A1
(43) Pub. Date: Apr. 10, 2025

(54) THROMBUS REMOVAL DEVICE

(71) Applicant: Chien Hsun Cheng, New Taipei City (TW)

(72) Inventor: Chien Hsun Cheng, New Taipei City (TW)

(21) Appl. No.: 18/482,028

(22) Filed: Oct. 6, 2023

(51) Int. Cl. A61B 17/3207; A61B 1/00; A61B 1/04; A61B 17/32

(52) U.S. Cl. CPC A61B 17/3207; A61B 1/00006; A61B 1/00087; A61B 1/00156; A61B 1/041; A61B 2017/320008; A61B 2017/32006

(57) ABSTRACT

A thrombus removal device, comprises a shell, a window, a propulsion device, a propulsion motor, a camera lens, a left thrombus scraper, a right thrombus scraper, a drive module, a drive motor, a control module, and a power supply module. The left and right thrombus scrapers are designed to remove thrombi adhered to the inner walls of blood vessels. With the integration of the camera lens, medical professionals can monitor the internal conditions of the blood vessels in real-time during the treatment process. This enhances the precision of thrombus removal and ensures the safety of the vascular structure, thereby reducing the risk of accidental injuries. Furthermore, the propulsion device enables the thrombus removal device to autonomously navigate within the blood vessels. This not only alleviates the manual operation workload of medical personnel but also optimizes the operational process and reduces procedure time, effectively enhancing overall operational efficiency and convenience.

### TECHNICAL FIELD

The present invention pertains to the field of medical devices, specifically to a thrombus removal device capable of scraping thrombi adhered to the inner walls of blood vessels.

### BACKGROUND

The formation of thrombi within blood vessels is a common and perilous medical condition that can lead to severe consequences such as stroke and myocardial infarction. In addressing these thrombi, the medical community has continuously endeavored to develop more effective methods and devices to eliminate thrombi adhering to the inner walls of vessels, thus restoring normal blood flow.

Traditional thrombi treatment methods primarily involve pharmacological treatments and surgical interventions. Pharmacological treatments typically encompass anticoagulants, which prevent blood coagulation and reduce the formation of new thrombi. However, prolonged use of anticoagulants may heighten the risk of bleeding, potentially leading to excessive hemorrhage in certain cases. Surgical interventions, on the other hand, usually require the incision of a patient's vessels to manually remove the thrombi. While surgery can ensure the patency of vessels, it inherently carries significant risks and necessitates extended recovery periods. Furthermore, it might result in pain for the patient and an increased risk of post-operative complications.

Consequently, traditional methods might fall short of delivering optimal therapeutic outcomes in some situations. There is a pressing need for a more efficient, safe, and side-effect-minimized thrombi treatment method to offer patients better and safer therapeutic options.

In light of the above, the aim of the present invention is to overcome the aforementioned challenges by introducing a thrombus removal device that can autonomously navigate within blood vessels, scrape thrombi adhered to the vessel walls, and provide real-time visualization of the inner vessel environment during the treatment process. This ensures both precision in thrombi removal and safety concerning the vessel's structural integrity, ultimately delivering enhanced therapeutic outcomes for patients and rectifying the deficiencies of conventional thrombi treatment methods.

### SUMMARY OF THE INVENTION

The aim of this invention is to overcome the shortcomings of the existing technology by introducing a thrombus removal device capable of autonomously navigating within blood vessels, scraping thrombi adhered to the vessel walls, and providing real-time visualization of the inner vessel environment during the treatment process. This ensures both precision in thrombi removal and safety concerning the vessel's structural integrity, ultimately delivering enhanced therapeutic outcomes for patients.

To address the above-mentioned problems and achieve the objectives of this invention, the technical means of this invention is realized as follows:

A thrombus removal device comprising: a shell internally equipped with an upper chamber, a lower chamber connected to the upper chamber, a left track positioned on a left end of the shell and connected to the lower chamber, and a right track positioned on the right end of the shell and connected to the upper chamber; a window positioned on a top surface of the shell and in communication with the upper chamber; a propulsion device situated on a rear end of the shell for advancing the shell within a blood vessel; a propulsion motor located within the upper chamber and linked to the propulsion device to drive the propulsion device; a camera lens situated within the upper chamber with the shooting direction of camera lens facing the window to capture images inside the blood vessel; a left thrombus scraper located within the left track, including a first drive wheel at a rear end of the left track, a first driven wheel at a front end of the left track driven by the first drive wheel, a first chain looped around and meshing with the first drive wheel and the first driven wheel, and a plurality of first scraper blades spaced on one side of the first chain protruding from the shell and driven by the first chain to scrape thrombi adhered to the inner wall of the blood vessel; a right thrombus scraper located within the right track, including a second drive wheel at a rear end of the right track, a second driven wheel at a front end of the right track driven by the second drive wheel, a second chain looped around and meshing with the second drive wheel and the second driven wheel, and a plurality of second scraper blades spaced on one side of the second chain protruding from the shell and driven by the second chain to scrape thrombi adhered to the inner wall of the blood vessel; a drive module positioned between the left and right thrombus scrapers and interfaced with the first drive wheel and the second drive wheel to facilitate the rotation of the first chain and the second chain; a drive motor connected to the drive module for actuating the rotation of the drive module, simultaneously driving the first chain and the second chain to rotate in both clockwise and counter-clockwise directions; a control module located in the lower chamber to initiate or halt the operation of the propulsion device, camera lens, left thrombus scraper, and right thrombus scraper; and at least one power supply module electrically connected to the propulsion motor, camera lens, drive motor, and control module to provide the necessary power for operation.

More preferably, wherein the rear end of the shell further includes a flow guide cover positioned over the propulsion device and equipped with a plurality of flow guide holes.

More preferably, wherein the propulsion device is composed of a propeller connected to the propulsion motor and rotates upon receiving power from the propulsion motor.

More preferably, wherein the shell includes an upper shell capable of accommodating the propulsion motor, the camera lens, the left thrombus scraper and the right thrombus scraper, and a lower shell positioned at the bottom end of the upper shell designed to house the control module and the power supply module.

More preferably, wherein the drive module includes a drive wheel assembly which is connected to the drive motor and interfaces with the first driving wheel and the second driving wheel, designed to receive power from the drive motor and to rotate accordingly.

More preferably, wherein the control module includes a circuit board electrically connected to the power supply module, a wireless receiver electrically connected to the circuit board, and a wireless transmitter electrically connected to the circuit board. The wireless receiver is capable of receiving control signals from a remote mobile device, and based on the signals, can remotely control the thrombus removal device.

More preferably, wherein a glass is also set at the position of the window.

More preferably, wherein the shell is bullet-shaped.

More preferably, wherein the first scraper and the second scraper are either arc-shaped or in the shape of the number “7”.

Compared with the prior art, the present invention can achieve the following functions and benefits:

Firstly, this invention, through the left thrombus scraper and the right thrombus scraper, can effectively scrape off thrombi adhered to the inner wall of blood vessels, thereby cleaning the vascular inner wall, which is conducive to the unblocking of vessels and the restoration of blood flow.

Secondly, by using a camera lens, medical professionals can observe the condition inside the blood vessel in real-time during the treatment, thus enhancing the precision of thrombus removal. At the same time, it ensures the safety of the vascular structure, reducing the risk of accidental damage.

Thirdly, the invention, with the propulsion device, can move autonomously within the blood vessel, alleviating the manual operation burden on medical staff. It also optimizes the operational process, shortens the operation time, and effectively enhances the overall operational efficiency and convenience.

Fourthly, this invention adopts a bullet-shaped shell, enabling the thrombus removal device to move with reduced resistance within the blood vessel, thereby improving the efficiency and smoothness of movement inside the vessel.

Fifthly, through the control module, medical professionals can remotely operate and monitor the thrombus removal device, such as activating the propulsion device, controlling the rotation direction of the first and second scraper blades, adjusting the angle of the camera lens, etc., thus enhancing the flexibility and convenience of operation.

### BRIEF DESCRIPTION OF THE DRAWINGS

FIG. 1 is a three-dimensional schematic view of the present invention;

FIG. 2 is an exploded schematic view of the present invention;

FIG. 3 presents another angle of the exploded schematic view of the invention;

FIG. 4 depicts a sectional schematic view of the present invention;

FIGS. 5 and 6 illustrate sectional implementation schematic views of the present invention.

### DETAILED DESCRIPTION OF THE EMBODIMENTS

With reference to the accompanying drawings and embodiments, a more detailed description of the present invention is provided below.

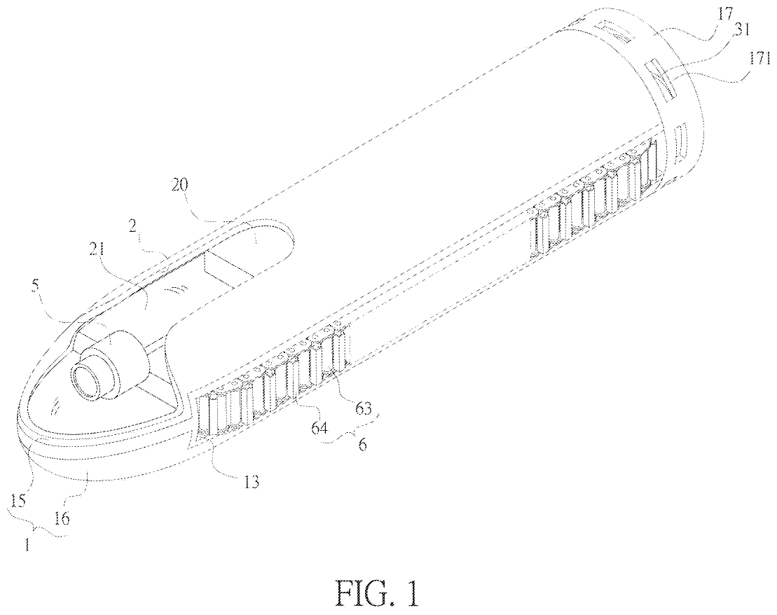

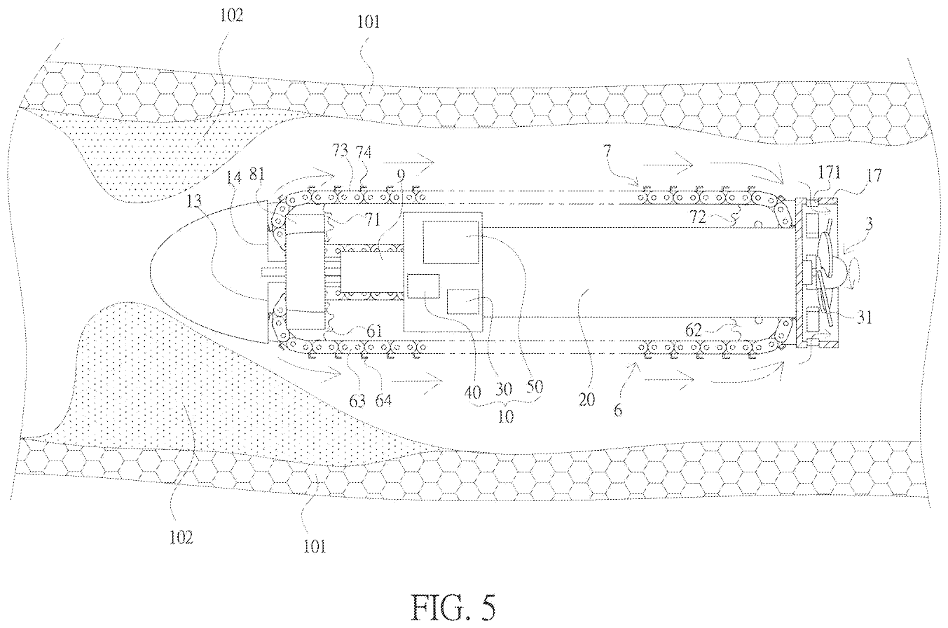

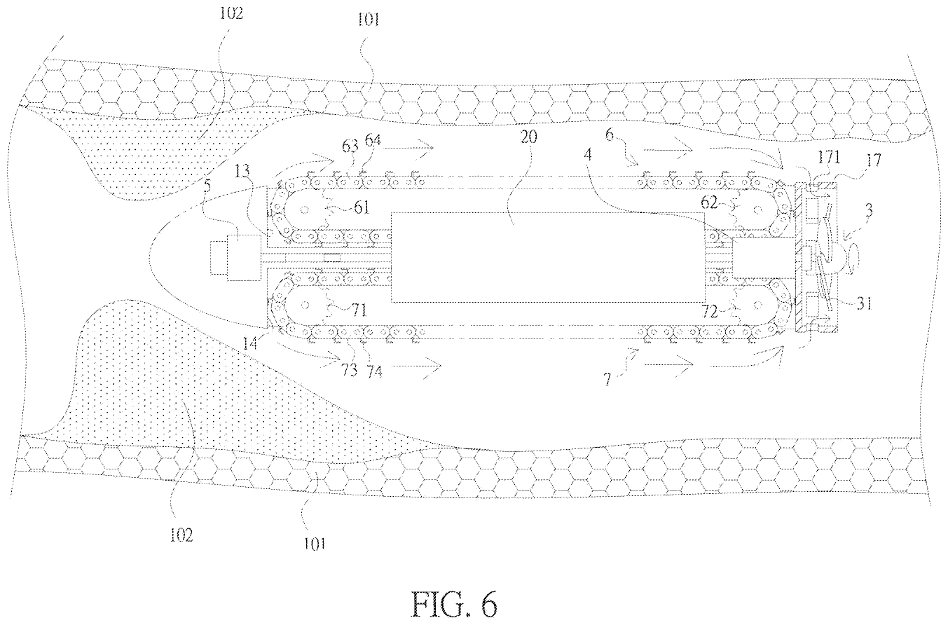

Referring to FIGS. 1 to 6, a thrombus removal device is disclosed, comprising: a shell (1), a window (2), a propulsion device (3), a propulsion motor (4), a camera lens (5), a left thrombus scraper (6), a right thrombus scraper (7), a drive module (8), a drive motor (9), a control module (10), and at least one power supply module (20). Each component will be described in detail as follows:

Referring to FIGS. 1 to 3, the shell (1) has an internal upper chamber (11), a lower chamber (12) communicating with the upper chamber (11), a left track (13) situated at the left end of the shell (1) connecting with the lower chamber (12), and a right track (14) situated at the right end of the shell (1) connecting with the upper chamber (11). Moreover, the shell (1) comprises an upper shell (15) suitable for housing the propulsion motor (4), the camera lens (5), the left thrombus scraper (6), and the right thrombus scraper (7), along with a lower shell (16) situated at the bottom of the upper shell (15), designed to accommodate the control module (10) and the power supply module (20).

The shell (1) is bullet-shaped, allowing the thrombus removal device to move within the blood vessel (101) with reduced resistance, thus enhancing its movement efficiency and fluidity within the blood vessel (101).

Further, the rear end of the shell (1) is also equipped with a flow guide cover (17) placed over the propulsion device (3) and fitted with multiple flow guide holes (171). The application of the flow guide cover (17) can prevent the risk of the propeller (31) coming into contact with the inner wall of the blood vessel (101), ensuring no damage to the vessel. Moreover, through the design of these flow guide holes (171), the blood from the blood vessel (101) can pass through, guiding the thrombus removal device to maintain the correct position and direction while advancing, achieving precise guidance.

Referring to FIGS. 1 to 3, the window (2) is set on the top surface of the shell (1) and communicates with the upper chamber (11), and at the window (2) position, there is also a glass (21) installed. Through the window (2), medical professionals can use the camera lens (5) to monitor the condition inside the blood vessel (101) in real-time, ensuring safety during thrombus removal. Furthermore, the addition of the glass (21) at the window (2) not only provides a clear visual observation but also prevents blood or other substances from entering the upper chamber (11), ensuring the effective operation of the camera lens (5).

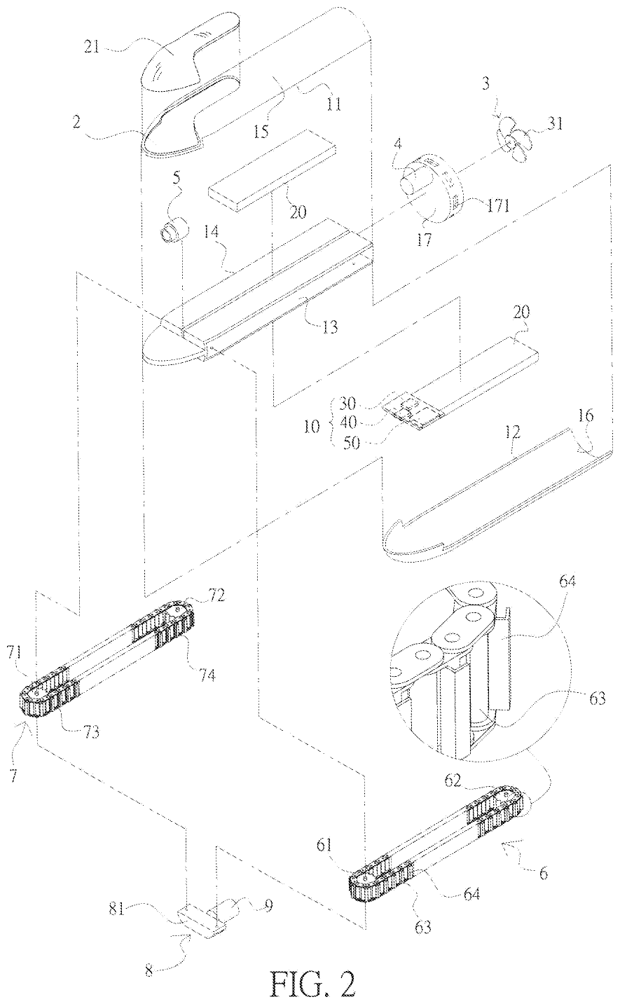

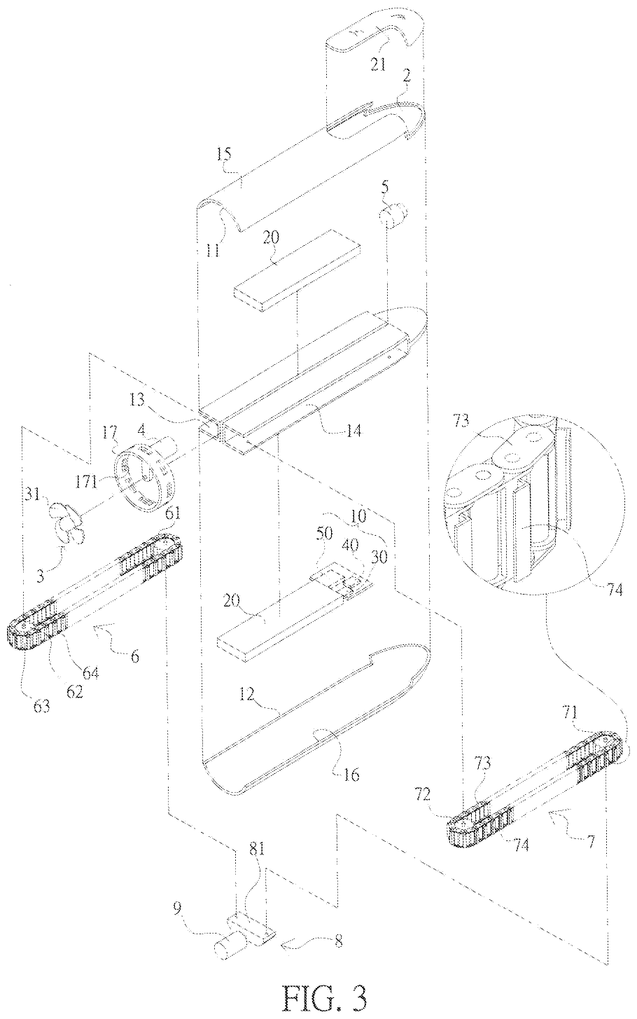

Referring to FIGS. 2 and 3, the propulsion motor (4) is positioned within the upper chamber (11) and connected to the propulsion device (3), ensuring the propulsion device (3) can move smoothly and accurately within the blood vessel (101), enhancing the stability of the operation.

Referring to FIGS. 2 to 4, the propulsion device (3) is located at the rear end of the shell (1) and serves to advance the shell (1) within the blood vessel (101). The propulsion device (3) is composed of a propeller (31) connected to the propulsion motor (4) and rotates upon receiving power from the propulsion motor (4). By utilizing the propulsion device (3), the thrombus removal device can autonomously navigate within the blood vessel (101), reducing the manual workload for medical personnel. This also optimizes the operational process, shortens the operation time, and effectively enhances the overall efficiency and convenience. Moreover, with the integration of the propeller (31), the propulsion device (3) can move steadily and continuously inside the blood vessel (101), avoiding abrupt accelerations or decelerations, reducing mechanical friction against the vessel's inner wall, and thereby minimizing the risk of damaging the blood vessel (101). This ensures safety and stability during treatment. Additionally, the continuous thrust generated by the movement of the propeller (31) also aids the left thrombus scraper (6) and the right thrombus scraper (7) in removing thrombi (102) adhered to the inner wall of the blood vessel (101), thus realizing superior scraping capability and effective thrombus removal.

Referring to FIGS. 1, 4, and 6, the camera lens (5) is positioned within the upper chamber (11), and its shooting direction faces the window (2), capturing images inside the blood vessel (101). Through this camera lens (5), medical professionals can observe the condition within the blood vessel (101) in real-time during the treatment process, thereby improving the accuracy of thrombus removal and ensuring safety, minimizing the risk of unintentional damage.

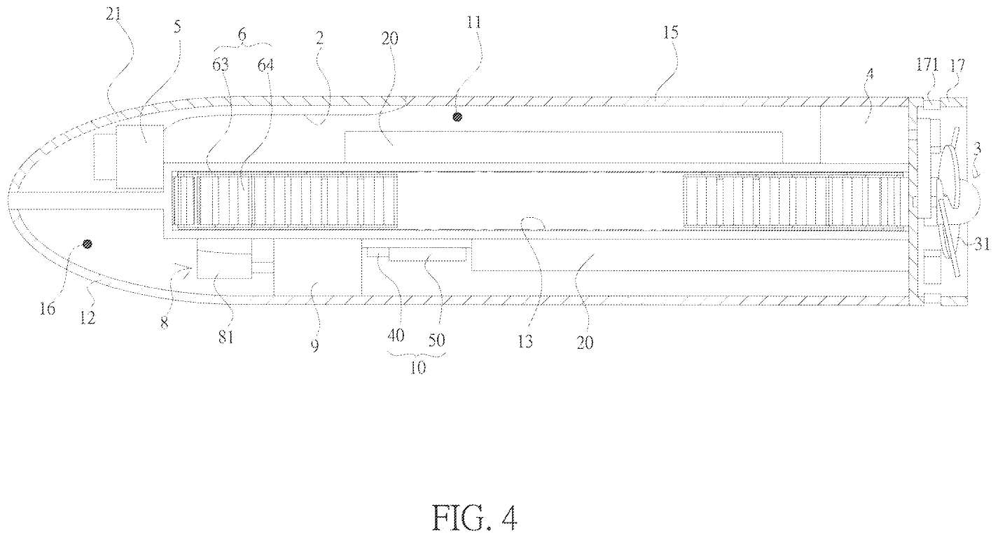

Referring to FIGS. 4 to 6, the left thrombus scraper (6) is positioned within the left track (13) and comprises a first drive wheel (61) located at the rear end of the left track (13), a first driven wheel (62) at the front end of the left track (13) which is driven by the first drive wheel (61), a first chain (63) looped around and engaging the first drive wheel (61) and the first driven wheel (62), and several first scraper blades (64) spaced on one side of the first chain (63) protruding from the shell (1) to scrape thrombi (102) adhered to the inner wall of the blood vessel (101). The right thrombus scraper (7) is positioned within the right track (14) and consists of a second drive wheel (71) at the rear end of the right track (14), a second driven wheel (72) at the front end of the right track (14) driven by the second drive wheel (71), a second chain (73) looped around and engaging the second drive wheel (71) and the second driven wheel (72), and several second scraper blades (74) spaced on one side of the second chain (73) protruding from the shell (1) to scrape thrombi (102) adhered to the inner wall of the blood vessel (101). Both the first scraper blade (64) and the second scraper blade (74) are either arc-shaped or shaped like the number “7”.

Furthermore, by utilizing the left thrombus scraper (6) and the right thrombus scraper (7), effective removal of thrombi (102) adhered to the inner wall of the blood vessel (101) can be achieved, thereby thoroughly cleaning the blood vessel (101) and promoting the restoration of blood flow. Additionally, the left thrombus scraper (6) and the right thrombus scraper (7) can be independently controlled in their rotational direction, allowing them not only to address single thrombus regions but also multiple thrombus areas, maximizing the comprehensiveness and efficiency of thrombus removal. Moreover, by connecting the first chain (63) and the second chain (73) to the first drive wheel (61), first driven wheel (62), second drive wheel (71), and second driven wheel (72), the first scraper blade (64) and the second scraper blade (74) can achieve continuous and stable rotation, preventing intermittent or vibrating motion. This ensures uniform coverage of the entire inner wall of the blood vessel (101) for efficient and comprehensive thrombus removal. Furthermore, by designing the first scraper blade (64) and the second scraper blade (74) in an arc shape or “7” shape, a closer contact with the inner wall of the blood vessel (101) is possible, reducing the chances of thrombus remnants and providing a more comprehensive thrombus removal effect.

Referring to FIGS. 2 to 4, the drive module (8) is positioned between the left and right thrombus scrapers (6,7), meshing with the first drive wheel (61) and the second drive wheel (71), driving the rotation of the first chain (63) and the second chain (73). The drive module (8) consists of a drive wheel assembly (81) connected to the drive motor (9), interfacing with the first drive wheel (61) and the second drive wheel (71), receiving power from the drive motor (9) to rotate. By positioning the drive module (8) between the left and right thrombus scrapers (6,7), synchronized operation is ensured during the scraping of thrombi (102), promoting uniform thrombus removal and reducing the risk of residual thrombi for precise therapeutic outcomes.

Referring to FIGS. 2 to 4, the drive motor (9) is connected to the drive module (8) to power its rotation and simultaneously drives both the first chain (63) and the second chain (73) to rotate in both clockwise and counter-clockwise directions. With the drive motor (9), the left thrombus scraper (6) can rotate counter-clockwise, and the right thrombus scraper (7) can rotate clockwise, allowing medical professionals to flexibly choose the rotation direction based on various conditions for targeted thrombus (102) removal. This minimizes the risk of damaging the inner wall of the blood vessel (101) and facilitates adaptive operation regardless of the blood vessel's (101) shape, whether curved, narrow, or intricate.

Referring to FIGS. 2 to 4, the control module (10) is located in the lower chamber (12) and is responsible for initiating or halting the operation of the propulsion device (4), camera lens (5), left thrombus scraper (6), and right thrombus scraper (7). The control module (10) includes a circuit board (30) electrically connected to the power supply module (20), a wireless receiver (40) electrically connected to the circuit board (30), and a wireless transmitter (50) also electrically connected to the circuit board (30). The wireless receiver (40) can receive control signals from a remote mobile device, enabling remote control of the thrombus removal device based on the signals.

Through the control module (10), medical professionals can remotely operate and monitor the thrombus removal device, such as activating the propulsion device (3), controlling the rotation direction of the first scraper blade (64) and the second scraper blade (74), adjusting the angle of the camera lens (5), etc., enhancing the flexibility and convenience of the operation.

Referring to FIGS. 2 to 4, the power supply module (20) is situated within the lower chamber (12) and is electrically connected to the propulsion motor (4), camera lens (5), drive motor (9), and the control module (10) to provide the necessary electrical power for operation. By positioning the power supply module (20) within the lower chamber (12), power management can be simplified. This design eliminates the need for multiple power supply interfaces or a complex connection process, reducing the risk of connection errors and maintenance complexity. It streamlines power management, making maintenance and servicing quicker and more convenient.

The detailed description of the structure, features, and operation effects of the present invention has been given above based on the illustrated embodiment. However, the description provided is merely the preferred embodiment of the present invention. The scope of the invention is not limited to the drawings. Thus, any modifications that conform to the spirit of the present invention, as long as they fall within the scope of equivalent effects, should be considered within the patent scope of this invention.

I claim:

1. A thrombus removal device comprising:
a shell (1) internally equipped with an upper chamber (11), a lower chamber (12) connected to the upper chamber (11), a left track (13) positioned on a left end of the shell (1) and connected to the lower chamber (12), and a right track (14) positioned on the right end of the shell (1) and connected to the upper chamber (11);
a window (2) positioned on a top surface of the shell (1) and in communication with the upper chamber (11);
a propulsion device (3) situated on a rear end of the shell (1) for advancing the shell (1) within a blood vessel (101);
a propulsion motor (4) located within the upper chamber (11) and linked to the propulsion device (3) to drive the propulsion device (3);
a camera lens (5) situated within the upper chamber (11) with the shooting direction of camera lens (5) facing the window (2) to capture images inside the blood vessel (101);
a left thrombus scraper (6) located within the left track (13), including
a first drive wheel (61) at a rear end of the left track (13),
a first driven wheel (62) at a front end of the left track (13) driven by the first drive wheel (61),
a first chain (63) looped around and meshing with the first drive wheel (61) and the first driven wheel (62), and
a plurality of first scraper blades (64) spaced on one side of the first chain (63) protruding from the shell (1) and driven by the first chain (63) to scrape thrombi (102) adhered to the inner wall of the blood vessel (101);
a right thrombus scraper (7) located within the right track (14), including
a second drive wheel (71) at a rear end of the right track (14),
a second driven wheel (72) at a front end of the right track (14) driven by the second drive wheel (71),
a second chain (73) looped around and meshing with the second drive wheel (71) and the second driven wheel (72), and
a plurality of second scraper blades (74) spaced on one side of the second chain (73) protruding from the shell (1) and driven by the second chain (73) to scrape thrombi (102) adhered to the inner wall of the blood vessel (101);
a drive module (8) positioned between the left and right thrombus scrapers (6,7) and interfaced with the first drive wheel (61) and the second drive wheel (71) to facilitate the rotation of the first chain (63) and the second chain (73);
a drive motor (9) connected to the drive module (8) for actuating the rotation of the drive module (8), simultaneously driving the first chain (63) and the second chain (73) to rotate in both clockwise and counter-clockwise directions;
a control module (10) located in the lower chamber (12) to initiate or halt the operation of the propulsion device (4), camera lens (5), left thrombus scraper (6), and right thrombus scraper (7); and
at least one power supply module (20) electrically connected to the propulsion motor (4), camera lens (5), drive motor (9), and control module (10) to provide the necessary power for operation.

2. The thrombus removal device as claimed in claim 1, wherein the rear end of the shell (1) further includes a flow guide cover (17) positioned over the propulsion device (3) and equipped with a plurality of flow guide holes (171).

3. The thrombus removal device as claimed in claim 1, wherein the propulsion device (3) is composed of a propeller (31) connected to the propulsion motor (4) and rotates upon receiving power from the propulsion motor (4).

4. The thrombus removal device as claimed in claim 1, wherein the shell (1) includes an upper shell (15) capable of accommodating the propulsion motor (4), the camera lens (5), the left thrombus scraper (6) and the right thrombus scraper (7), and a lower shell (16) positioned at the bottom end of the upper shell (15) designed to house the control module (10) and the power supply module (20).

5. The thrombus removal device as claimed in claim 1, wherein the drive module (8) includes a drive wheel assembly (81) which is connected to the drive motor (9) and interfaces with the first driving wheel (61) and the second driving wheel (71), designed to receive power from the drive motor (9) and to rotate accordingly.

6. The thrombus removal device as claimed in claim 1, wherein the control module (10) includes a circuit board (30) electrically connected to the power supply module (20), a wireless receiver (40) electrically connected to the circuit board (30), and a wireless transmitter (50) electrically connected to the circuit board (30). The wireless receiver (40) is capable of receiving control signals from a remote mobile device, and based on the signals, can remotely control the thrombus removal device.

7. The thrombus removal device as claimed in claim 1, wherein a glass (21) is also set at the position of the window (2).

8. The thrombus removal device as claimed in claim 1, wherein the shell (1) is bullet-shaped.

9. The thrombus removal device as claimed in claim 1, wherein the first scraper (64) and the second scraper (74) are either arc-shaped or in the shape of the number “7”.
